(19) United States
(12) Patent Application Publication
(10) Pub. No.: US 2024/0352442 A1
(43) Pub. Date: Oct. 24, 2024

(54) METHOD FOR ATTENUATING INFLUENZA VIRUS BY SYNONYMOUS MUTATION AND DELETION MUTATION, AND ATTENUATED INFLUENZA VIRUS STRAIN AND USE

(71) Applicant: Zhejiang Difference Biological Technology Co., Ltd., Hangzhou (CN)

(72) Inventors: Shuihua MAO, Hangzhou (CN); Mengyun ZHOU, Hangzhou (CN); Ruiting CHEN, Hangzhou (CN); Xiaofei LIU, Hangzhou (CN); Jiasheng SONG, Hangzhou (CN)

(21) Appl. No.: 18/137,786

(22) Filed: Apr. 21, 2023

(51) Int. Cl. C12N 15/10; C12N 7/04

(52) U.S. Cl. CPC C12N 15/102; C12N 7/04; C12N 2760/16121

(57) ABSTRACT

The present disclosure relates to a method for attenuating an influenza virus, an attenuated influenza virus strain and use thereof, and belongs to the technical field of attenuated vaccines. In the present disclosure, the method includes the following steps: conducting synonymous mutation on an overlap of an M2 gene and an M1 gene in an M gene of an influenza A virus, while ensuring integrity and invariance of an amino acid sequence encoded by the M1 gene; conducting mutation into termination codon and deletion of partial nucleotide sequence in a transmembrane domain of the M2 gene, and rescuing an attenuated influenza virus strain using a reverse genetic system.

### REFERENCE TO SEQUENCE LISTING

A computer readable XML file entitled “HLP20230402520_seqlist.xml”, that was created on May 8, 2023, with a file size of about 27,702 bytes, contains the sequence listing for this application, has been filed with this application, and is hereby incorporated by reference in its entirety.

### TECHNICAL FIELD

The present disclosure relates to the technical field of attenuated vaccines, in particular to a method for attenuating an influenza virus, an attenuated influenza virus strain and use thereof.

### BACKGROUND

Influenza viruses belong to the single-stranded and negative-sense segmented RNA viruses in the Orthomyxoviridae family, and have a high degree of genetic drift. Influenza viruses break out almost every year, leading to the annual need for vaccine production, research and development based on the epidemic strains in current seasons. The attenuated live vaccines of influenza virus are quick and convenient to produce, and can cause mucosal immunity to prevent virus infection and transmission compared with inactivated vaccines.

Influenza virus is a segmented negative-strand RNA virus whose genome mainly includes eight segments: PB1, PB2, PA, HA, NP, NA, M, and NS. M gene encodes two proteins, M1 and M2, through alternative splicing. M1, a structural protein of influenza virus, forms a protein envelope layer under the virus cyst membrane, and is related to virus assembly and budding. The M2 is highly conserved in influenza A. M2 has ion channel activity and functions in the early stages of a virus life cycle, namely, the virus penetration and uncoating stages. Moreover, this protein is involved in virus assembly and morphogenesis, and can be used as a research point for virus attenuation. However, at present, there is still a lack of highly-effective methods for attenuating influenza viruses targeting the M gene.

### SUMMARY

An objective of the present disclosure is to provide a method for attenuating an influenza virus by synonymous mutation, deletion, and mutation into termination codon on an M gene, and an attenuated influenza virus strain and use. In the present disclosure, the attenuated influenza virus strain obtained by the method has the following advantages. 1) The proliferation process is so stable that the virus is not likely to return to a wild type. 2) The virus cannot proliferate in MDCK cells inoculated at low dosages, but can grow and reproduce in the MDCK cells inoculated at high doses, reflecting the restricted proliferation of the attenuated virus. The strain has greater potential for inducing immunity than that of single replication defective viruses. 3) This strain has desirable safety and high immune effect, and can be used as a candidate strain of attenuated live vaccines. 4) This strain can be inoculated into SPF chicken embryos and grows and reproduces in the chicken embryos, thereby bringing a great convenience to production practice. 5) In the present disclosure, the method is applicable to subtypes of the influenza A virus, and is an important way to attenuate the influenza A virus.

The present disclosure provides a method for attenuating an influenza virus by deletion, synonymous mutation and mutation into termination codon, including the following steps: conducting synonymous mutation on an overlap of an M2 gene and an M1 gene in an M gene of an influenza A virus, while ensuring integrity and invariance of an amino acid sequence encoded by the M1 gene; conducting mutation into termination codon and deletion of partial nucleotide sequence in a transmembrane domain of the M2 gene, and rescuing an attenuated influenza virus strain using a reverse genetic system.

In some embodiments, a background strain of the method includes A/Puerto Rico/8/1934.

In some embodiments, the synonymous mutation includes: conducting mutation on the 715th base to the 760th base of the M gene into a nucleotide sequence of SEQ ID NO: 1.

In some embodiments, the mutation into termination codon includes: conducting mutation on the 761th base to the 766th base of the M gene into a nucleotide sequence of TGATGA.

In some embodiments, the deletion of partial nucleotide sequence includes: conducting deletion on a nucleotide sequence of any position and length from the 767th base to the 877th base of the M gene.

In some embodiments, the deletion of partial nucleotide sequence specifically includes:
conducting deletion on the 767th base to the 774th base of the M gene:
conducting deletion on the 767th base to the 794th base of the M gene:
conducting deletion on the 767th base to the 817th base of the M gene:
conducting deletion on the 767th base to the 839th base of the M gene: or
conducting deletion on the 767th base to the 877th base of the M gene.

The present disclosure further provides an attenuated influenza virus strain prepared by the method.

In some embodiments, the attenuated influenza virus strain takes the A/Puerto Rico/8/1934 as a background strain, and the M gene is subjected to modifications including the synonymous mutation, the mutation into termination codon, and the deletion of partial nucleotide sequence; and the M gene of a modified influenza virus has a nucleotide sequence set forth in one of SEQ ID NO: 2 to SEQ ID NO: 6.

The present disclosure further provides a set of defective plasmids for preparing the attenuated influenza virus strain, where the defective plasmids include the M gene of the influenza virus with the deletion and the mutation including the synonymous mutation and the mutation into termination codon.

The present disclosure further provides use of the method or the attenuated influenza virus strain or the defective plasmids in the preparation and production of an attenuated vaccine for influenza.

The present disclosure provides a method for attenuating an influenza virus by synonymous mutation and deletion as well as mutation into termination codon. In the present disclosure, the synonymous mutation in the overlap region leads to changes in the reading frames of M2 gene, and thus to changes in amino acids encoded by the M2 gene. The mutation into termination codons terminate the translation after the ectodomain, leading to incomplete M2 protein. Together with the mutation into termination codon, the synonymous mutation affects the function of the M2 protein at amino acid level. Deletion of partial sequence of M2 further prevents the M gene from returning to a wild type. Simultaneously, the synonymous mutation and deletion, together with the mutation into termination codon, greatly affect the secondary RNA structure of the M gene, thereby affecting the biological properties of the rescued virus. In this way, a series of attenuated influenza virus strains is produced. In addition, different from a previous influenza virus attenuating method, the attenuated influenza virus strain produced by the present disclosure has the following advantages. 1) The strain can hardly revert to a wild type. 2) The virus cannot proliferate in MDCK cells inoculated at low dosages, but can grow and reproduce in the MDCK cells inoculated at high doses, reflecting the attenuated virus-restricted proliferation ability. The strain has greater potential for inducing immunity than that of single replication defective viruses. 3) Experiments in mice have shown that the attenuated influenza virus has a desirable safety and a high immunogenicity, and can be used as a candidate strain of attenuated live vaccines. 4) This strain can grow well on chicken embryos, and provides a possibility for production of the virus in chicken embryos.

### BRIEF DESCRIPTION OF THE DRAWINGS

FIG. 1 shows a growth curve of the M-defective influenza virus provided by the present disclosure;

FIG. 2 shows a proliferation result of the M-defective influenza virus in the MDCK cells provided by the present disclosure:

FIG. 3 shows a body weight change of mice provided by the present disclosure after being inoculated with a virus:

FIG. 4 shows a virus titer of the turbinate and the lung lobe tissue of mice provided by the present disclosure three days after being inoculated with the virus:

FIG. 5 shows a pathological section of the lung lobe tissue of mice provided by the present disclosure three days after being inoculated with the virus; and

FIG. 6 shows a body weight change of immunized mice provided by the present disclosure 10 d after challenge.

### DETAILED DESCRIPTION OF THE EMBODIMENTS

The present disclosure provides a method for attenuating an influenza virus by deletion, synonymous mutation and mutation into termination codon, including the following steps:

conducting synonymous mutation on an overlap of an M2 gene and an M1 gene in an M gene of an influenza A virus, while ensuring integrity and invariance of an amino acid sequence encoded by the M1 gene; conducting mutation into termination codon and deletion of partial nucleotide sequence in a transmembrane domain of the M2 gene, and rescuing an attenuated influenza virus strain using a reverse genetic system.

In the present disclosure, the synonymous mutation in the M gene (an overlap of the M2 gene and the M1 gene) results in changes in the reading frame of M2 gene, and thus in changes in amino acids encoded by the M2 gene. The synonymous mutation is combined with deletion and mutation into termination codon to change an RNA sequence at a base level, thereby further affecting a secondary structure of the RNA. Meanwhile, 2 termination codons obtained by the mutation terminate the translation in and after the transmembrane region, producing incomplete M2 protein. In this way, a series of attenuated influenza virus strains is produced. The attenuated influenza virus strain prepared by the attenuating method can hardly return to a wild type: moreover, the strain can grow well on MDCK cells or chicken embryos, which provides a possibility for production of the virus in chicken embryos or MDCK cells. Moreover, experiments on mice show that the attenuated influenza virus strain is safe on the mice, thereby laying a foundation for the production of safe and effective attenuated influenza vaccines.

In the present disclosure, the background strain of the method includes preferably A/Puerto Rico/8/1934.

In the present disclosure, the synonymous mutation includes preferably: conducting mutation on the 715th base to the 760th base of the M gene into a nucleotide sequence of SEQ ID NO: 1, a Specifically, sequence GCCTATCAGAAACGAATGGGGGT GCAGATGCAACGGTTCAAGTGAT (SEQ ID NO: 17) is mutated into the sequence GCGTACCAAAAGCGTATGGGTGTTCAAATGCAGAGATTTAAATAAG (SEQ ID NO: 1) to form an M(sn)-defective plasmid.

In the present disclosure, the mutation into termination codon includes preferably: conducting mutation on the 761th base to the 766th base of the M gene into a nucleotide sequence of TGATGA. That is, two termination codons are introduced, specifically, the 761st base to the 766th base CCTCTC is mutated into TGATGA.

In the present disclosure, the deletion of partial nucleotide sequence includes preferably: conducting deletion on a nucleotide sequence of any position and length from the 767th base to the 877th base of the M gene. More preferably, the deletion of partial nucleotide sequence specifically includes:
conducting deletion on the 767th base to the 774th base of the M gene:
conducting deletion on the 767th base to the 794th base of the M gene:
conducting deletion on the 767th base to the 817th base of the M gene:
conducting deletion on the 767th base to the 839th base of the M gene: or conducting deletion on the 767th base to the 877th base of the M gene.

Specifically; the 767th base to the 774th base ACTATTGC of the M gene is deleted: after combination of the synonymous mutation and the mutation into termination codon, an M(sn)+Mut6+Del8-defective plasmid is formed:
the 767th base to the 794th base ACTATTGCCGCAAATATCATTGGGATCT (SEQ ID NO: 18) of the M gene is deleted: after combination of the synonymous mutation and the mutation into termination codon, an M(sn)+Mut6+Del28-defective plasmid is formed;
the 767th base to the 817th base ACTATTGCCGCAAATATCATTGGGATCTTGCACTTGACATTGTGGATTCTT (SEQ ID NO: 19) of the M gene is deleted: after combination of the synonymous mutation and the mutation into termination codon, an M(sn)+Mut6+Del51-defective plasmid is formed:
the 767th base to the 839th base ACTATTGCCGCAAATATCATTGGGATCTTGCACTTGACATTGTGGATTCTTGATCGTCT TTTTTTCAAATGCA (SEQ ID NO: 20) of the M gene is deleted: after combination of the synonymous mutations and the mutation into termination codon, an M(sn)+Mut6+Del73-defective plasmid is formed: or
the 767th base to 877th base the ACTATTGCCGCAAATATCATTGGGATCTTGCACTTGACATTGTGGATTCTTGATCGTCT TTTTTTCAAATGCATTTACCGTCGCTTTAAATACGGACTGAAAGGAGGGCCT (SEQ ID NO: 21) of the M gene is deleted: after combination of the synonymous mutation and the mutation into termination codon, an M(sn)+Mut6+Del111-defective plasmid is formed.

In the present disclosure, an M gene-defective plasmid is preferably constructed according to the deletion, the synonymous mutation and the mutation into termination codon, and then the M gene-defective plasmid is co-transfected with other 7 plasmids (PB2. PB1. PA. NP. NS. HA, and NA) for reverse genetics of PR8 background influenza and a plasmid expressing a full-length M2 protein (PR8-M2) into cells, and a virus is harvested to obtain the attenuated influenza virus strain.

The present disclosure further provides an attenuated influenza virus strain prepared by the method. In the present disclosure, the attenuated influenza virus strain takes the A/Puerto Rico/8/1934 as a background strain, and modifications including the synonymous mutation, the mutation into termination codon, and the deletion of partial nucleotide sequence are conducted. A modified M gene of the influenza virus preferably has a nucleotide sequence set forth in one of SEQ ID NO: 2 to SEQ ID NO: 6.

The present disclosure further provides a set of defective plasmids for preparing the attenuated influenza virus strain, where the defective plasmids include the M gene of the influenza virus with the deletion, the synonymous mutation and the mutation into termination codon. The defective plasmid has a nucleotide sequence preferably set forth in one of SEQ ID NO: 2 to SEQ ID NO: 6. A primer for preparing the defective plasmid preferably has a nucleotide sequence of SEQ ID NO: 7 to SEQ ID NO: 16.

The present disclosure further provides use of the method or the attenuated influenza virus strain or the defective plasmids in the preparation and production of an attenuated influenza vaccine.

The method for attenuating an influenza virus by synonymous mutation in combination with deletion and mutation into stop codon, and the attenuated influenza virus strain and the use according to the present disclosure will be further described in detail below with reference to specific examples. The technical solutions of the present disclosure include, but are not limited to, the following examples.

### Example 1

### Construction of M Gene-Defective Plasmid

Primers M(sn)-F: atgggtgttcaaatgcagagatttaaataagcctctcactattgccgcaaat (SEQ ID NO: 22) and M(sn)-R: gagaggcttatttaaatctctg (SEQ ID NO: 23) were designed. A pFlu-PR8-M (the M gene has a nucleotide sequence of SEQ ID NO: 24) carrier plasmid in an 8-plasmid system of reverse genetics of influenza was used as a template, and a synonymous mutation sequence was amplified by PCR according to the instructions of PrimerSTAR. Gel electrophoresis was conducted to confirm that the size of the amplified sequence was correct, and a target fragment was recovered by cutting the gel, and then cloned by homologous recombination according to the instructions of the NEBuilder® HiFi DNA Assembly kit, so as to obtain an M(sn) plasmid. On the basis of the M(sn) plasmid, primers were designed for PCR amplification and homologous recombination to obtain an M(sn)+Mut6+Del8-defective plasmid, an M(sn)+Mut6+Del28-defective plasmid, an M(sn)+Mut6+Del51-defective plasmid, an M(sn)+Mut6+Del73-defective plasmid, and an M(sn)+Mut6+Del111-defective plasmid.

The primers for amplification are shown in Table 1:

TABLE 1
Primers for amplification

| Defective | | |
|---|---|---|
| plasmid | Primer | Sequence (SEQ ID NO.) |
| M(sn) + Mut6 + | F | tcaagtgattgatgacgcaaatatcat |
| Del8 | | tgggatcttgc (7) |
| | R | tcatcaatcacttgaaccgttg (8) |
| M(sn) + Mut6 + | F | tcaagtgattgatgatgcacttgacat |
| Del28 | | tgtggattcttg (9) |
| | R | tcatcaatcacttgaaccgttg (10) |
| M(sn) + Mut6 + | F | tcaagtgattgatgagatcgtcttttt |
| Del51 | | ttcaaatg (11) |
| | R | tcatcaatcacttgaaccgttg (12) |
| M(sn) + Mut6 + | F | tcaagtgattgatgatttaccgtcgct |
| Del73 | | ttaaatacg (13) |
| | R | tcatcaatcacttgaaccgttg (14) |
| M(sn) + Mut6 + | F | tcaagtgattgatgatctacggaagga |
| Del111 | | gtgccaaag (15) |
| | R | tcatcaatcacttgaaccgttg (16) |

### Example 2

### Rescue of Defective Influenza Virus

293T cells were plated into Thermo Fisher's special six-well plates, and transfected when the confluence reached 70% to 80%. Rescue of defective recombinant influenza virus was conducted by classical “6+2” influenza reverse genetic system. 0.5 μg for each of gene-defective plasmids of 6 PR8 internal genes pFlu-PR8-PB2, pFlu-PR8-PB1, pFlu-PR8-PA, pFlu-PR8-NP, pFlu-PR8-NS, and pFlu-PR8-M, as well as 2 external genes pFlu-PR8-HA and pFlu-PR8-NA, and 0.25 μg of the plasmid expressing full-length M2 were co-transfected into the 293T cells (Lipofectamine 3000). 24 h after transfection, a medium containing TPCK-Trypsin with a final concentration of 0.5 μg/ml was replaced. 48 h after transfection, the cell supernatant was collected and inoculated into 8-day-old SPF chicken embryos through an allantoic cavity at 0.2 ml/piece. The inoculated chicken embryos were incubated in a 37° C. incubator for 48 h. An allantoic fluid of the chicken embryos (F0 generation) was collected to attain the defective influenza viruses therein, and whether there was a hemagglutination value was determined. If there was no hemagglutination value, the virus was passed blindly for one generation, and then whether there was a hemagglutination value was determined again. The resulting M-detective influenza viruse strains were named as PR8-M(sn). PR8-M(sn)+Mut6+Del8. PR8-M(sn)+Mut6+Del28. PR8-M(sn)+Mut6+Del51. PR8-M(sn)+Mut6+Del73, and PR8-M(sn)+Mut6+Del111, respectively.

### Example 3

### Growth Curve of Virus

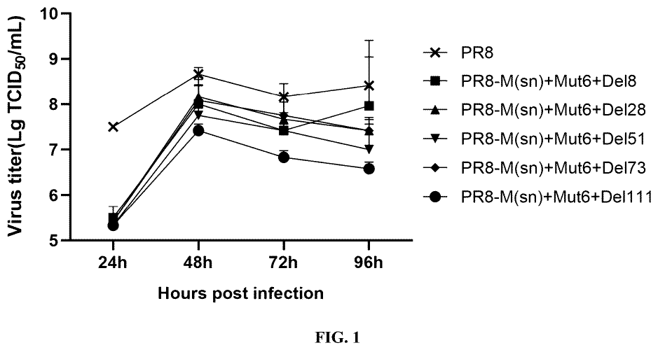

MDCK-M2 cells were plated in a 24-well plate, and after the cells had grown to a monolayer, the M-defective influenza virus strain was inoculated in triplicates into the cells at a multiplicity of infection (MOI) of 0.001. After 2 h of infection, a liquid in the 24-well plate was discarded, the cells were washed with PBS, and then DMEM medium containing 2% FBS was added to maintain cell growth, and the cells were cultured in an incubator at 37° C. and 5% CO₂. Viruses were harvested at 12 h. 24 h. 36 h. 48 h. 60 h. and 72 h after infection, respectively. The harvested virus liquids at different time points were subjected to serial tenfold dilution, with each dilution repeated 4 times, and inoculated into monolayer MDCK-M2 cells in a 96-well plate. After 2 h of infection, the medium was replaced with 2% FBS in DMEM to maintain cell growth. After 48 h. the cytopathic changes were observed, a virulence of the defective influenza strain was collected, and TCID₅₀ was calculated by a Reed-Muench method. After the data analysis was completed, a growth curve of the M-defective influenza virus was plotted, and the results were shown in FIG. 1.

All attenuated viruses had reduced growth ability compared to wild-type PR8 virus. 24 h after infection, the titers of each type of attenuated viruses were about 105 TCID₅₀/ml. However, the viruses then multiplied in large numbers. At 48 h after infection, the attenuated virus had a titer of around 10⁷ TCID₅₀/ml. It was seen that all the attenuated virus strains produced by the present disclosure could grow at high titer in the MDCK-M2 cells, thus meeting the normal production requirements.

### Example 4

### M-Defective Influenza Virus can Grow in Normal MDCK

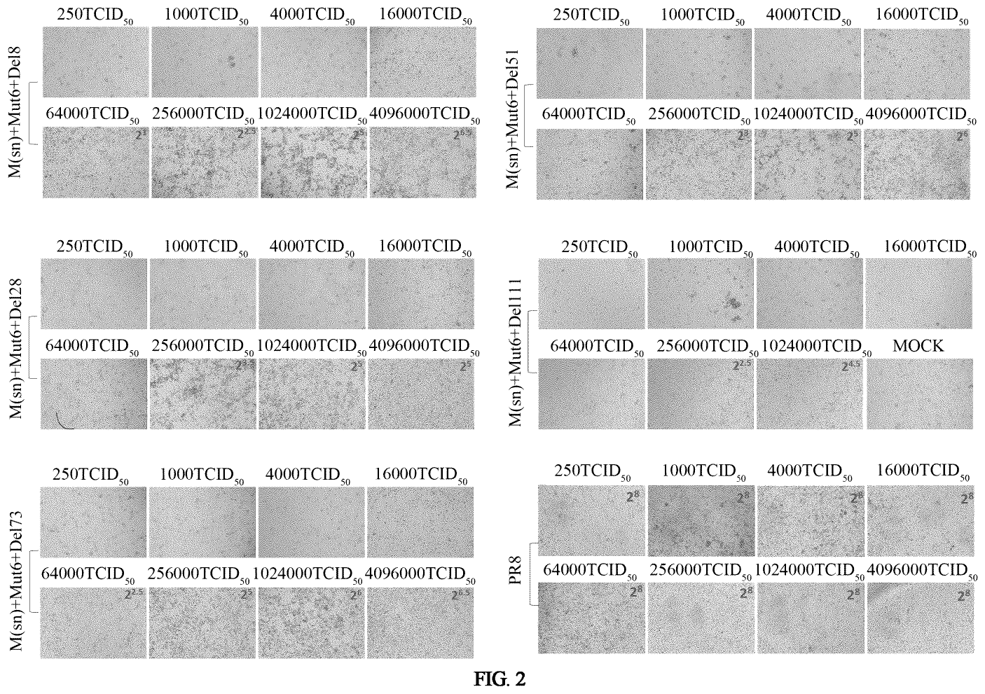

250, 1,000, 4,000, 16,000, 64,000, 256,000, 1,024,000, and 4,096,000 TCID₅₀ M-defective influenza viruses were inoculated into MDCK cells in a 48-well plate, and the cytopathic changes were observed and an HA titer of the cell culture medium was detected. The results are shown in FIG. 2. It was seen from the results that the M-defective influenza virus of the present disclosure could not proliferate when inoculated at low dosages into MDCK cells, but could grow and reproduce in the MDCK cells when inoculated at high dosages. This reflected the ability of the attenuated virus to proliferate in a restricted manner. The strain was advantageous for inducing better protection than that of single replication defective viruses.

### Example 5

### M-Defective Influenza Virus can Grow in Chicken Embryos

The M-defective influenza virus stocks were inoculated into 8 to 10-day-old SPF chicken embryos, at 100 μL/embryo, and three chicken embryos were inoculated with each strain of the virus. After 72 h, an allantoic fluid was harvested to detect the HA titer of the virus. Results are shown in Table 2.

TABLE 2
HA titer of chick embryo culture of M-defective influenza virus

| Attenuated virus strain | Survival state | HA | | |
|---|---|---|---|---|
| PR8-M (sn) + Mut6 + Del8 | Live, live, live | 9 | 9 | 8 |
| PR8-M (sn) + Mut6 + Del28 | Live, live, live | 8 | 8 | 9.5 |
| PR8-M (sn) + Mut6 + Del51 | Live, live, live | 7 | 5 | 6 |
| PR8-M (sn) + Mut6 + Del73 | Live, live, live | 9 | 8.5 | 8 |
| PR8-M (sn) + Mut6 + Del111 | Live, live, live | 8 | 8.5 | 9 |

It was seen from Table 2 that all M-defective influenza viruses could grow and reproduce in large quantities in chicken embryos, a highest titer of HA could reach more than 29.5, and averagely most strains could reach 28.5. In addition to higher production capacity, the culture of chicken embryos had simpler requirements for equipment and workshops than those for cell culture, which provides a great convenience for the production of defective influenza viruses.

### Example 6

### Passage Trial

The virus was continuously passaged for 10 generations in an MDCK-M2 cell line, and a stability of the M gene of the virus was detected by sequencing. After the detection, the M genes of all attenuated viruses remained unchanged relative to those before passage. It was seen that the attenuated influenza virus of the present disclosure had stable inheritance at the gene level.

### Example 7

Experiments with M-Defective Influenza Virus on Mice

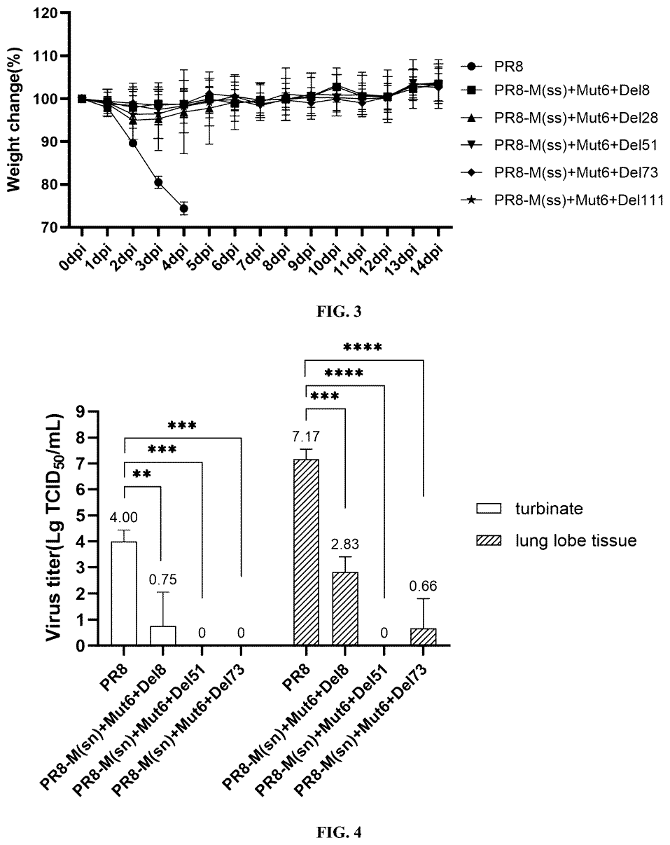

Female Balb/c mice aged 7 to 8 weeks were inoculated intranasally with the M-defective influenza virus and a wild-type PR8 strain, while the mice in a control group were given PBS, with 5 to 8 mice in each group. Body weight changes and infection symptoms of mice were recorded daily after inoculation (FIG. 3). 3 d after the inoculation, 3 mice in each group of PR8-M(sn)+Mut6+Del8, PR8-M(sn)+Mut6+Del51, and PR8-M(sn)+Mut6+Del73 were euthanized.

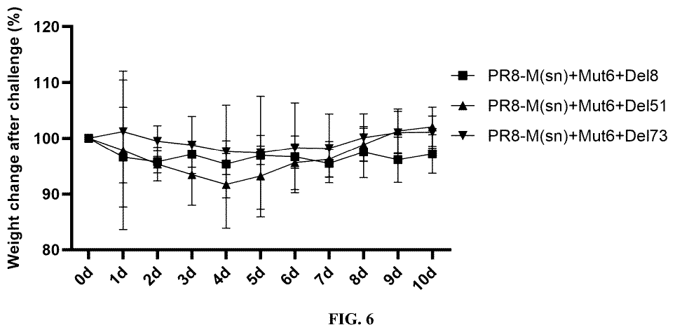

The lung and turbinate tissues were dissected for pathological detection and virus titer determination. The pathological detection included: embedding tissues in paraffin, conducting sectioning, bleaching, drying, and HE staining, followed by taking pictures. The method for detecting virus titer was as follows: the MDCK-M2 cells were spread on a 96-well plate. After the cells grew to a monolayer, the recombinant virus was serially diluted with DMEM by 10-fold ratio to 106. A culture medium in the 96-well plate was discarded, the well plate was washed with DMEM. 100 μL of a recombinant virus solution of corresponding dilution was added to each well, and 4 replicates were set for each gradient. The cells were incubated in a cell incubator at 37° C. and 5% CO₂ for 2 h. a supernatant was discarded, and the medium was replaced with a DMEM medium containing 2% FBS to maintain cell growth. After 48 h. a supernatant was discarded, and the well plate was washed twice with PBS. The fluorescence in each well was observed and recorded under an inverted fluorescence microscope, and the TCID₅₀ was calculated using a Reed-Muench method. Blood was collected 14 d after the first immunization, and the HI (hemagglutination inhibition) level of the mouse serum was detected. The method for detecting HI level was as follows: four-unit antigen was configured, where a dilution factor of virus antigen=virus agglutination value/4. A mouse serum to be tested was inactivated at 56° C. for 30 min in advance. 25 μL of the serum to be tested was added to a first well of the 96-well plate, the immune serum was serially diluted at a ratio of 1:2, 25 μL of the four-unit antigen was added to the diluted serum, and a reaction was allowed at room temperature for 30 min. 25 μL of a 1% chicken erythrocytes suspension was added to each well, the test results were observed after allowance to stand at room temperature for 30 min, and the highest dilution where hemagglutination occurred, namely the HI level was recorded. The results are shown in Table 3. 21 d after immunization, the mice were challenged with the PR8 virus at 106 TCID₅₀/50 μL. and the state and body weight of the mice were recorded (FIG. 6).

It was seen from the results that after being infected with the defective attenuated virus, the body weight of the mice decreased slightly, within 5% on 2nd and 3rd days, and then recovered. On the 5th day, all the inoculated mice except the control group had their body weight returned to the initial stage and then grew normally; and by the 14th day, the mice showed a desirable mental state. In contrast, all 5 mice in the control group inoculated with wild-type PR8 virus died on the 5th day. In addition. 3 d after inoculation, the viral titers of the wild-type PR8 strain were higher than those of the attenuated influenza virus in the turbinate and lung lobe tissues of mice. In lung tissue, the titer of wild-type virus was significantly higher than that of attenuated influenza viruses PR8-M(sn)+Mut6+Del8. PR8-M(sn)+Mut6+Del51, and PR8-M(sn)+Mut6+Del73 (FIG. 4). A small amount of PR8-M(sn)+Mut6+Del8 recombinant virus could still be detected in lung lobe tissues, which demonstrated that the attenuated strains produced by a same method were different in toxicity: In another aspect, the results of pathological sections showed that the PR8-M(sn)+Mut6+Del73 attenuated virus strain caused a small amount of diffuse lymphocyte infiltration around the bronchi in 2/3 lung lobes of the mouse lung tissue, and the pulmonary interstitium showed moderate fibrosis. The PR8-M(sn)+Mut6+Del8 caused diffuse lymphocyte infiltration around a small amount of bronchi in 1/3 of the lung lobe, and moderate to mild fibrosis was seen in the pulmonary interstitium. The PR8-M(sn)+Mut6+Del51 only caused diffuse lymphocyte infiltration around a small number of lung lobes. It was seen that the different attenuated strain induced different degree of pathology and sparked different immuneresponse strength. Among them, the PR8-M(sn)+Mut6+Del8 strain was relatively more toxic (FIG. 4).

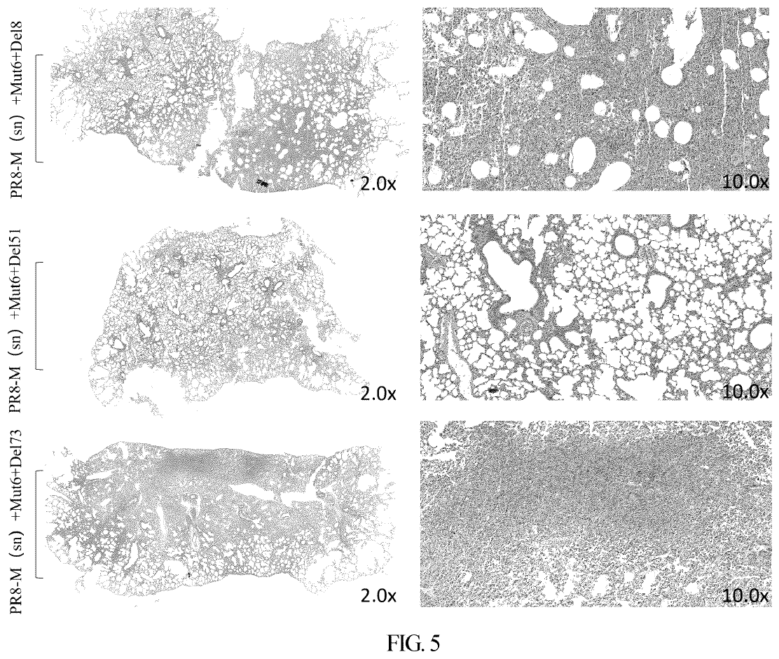

The above results in FIG. 3 and FIG. 4 showed that the defective influenza virus rescued by the present disclosure was generally safe in mice and did not cause death in mice. However, the viral titer post 3 days-inoculation and pathogenicity of the strains to mice were different. The strain with slower virus shedding was PR8-M(sn)+Mut6+Del8 comprehensively (FIGS. 4 and 5).

TABLE 3
Results of serum HI antibody

| Attenuated virus strain | HI |
|---|---|
| PR8-M (sn) + Mut6 + Del8 | 2⁸ |
| PR8-M (sn) + Mut6 + Del28 | 2⁴ |
| PR8-M (sn) + Mut6 + Del51 | 2⁶ |
| PR8-M (sn) + Mut6 + Del73 | 2⁷ |
| PR8-M (sn) + Mut6 + Del111 | 2⁶ |

14 d after the mice were infected with the virus, the serum HI antibodies were shown in Table 3. From the results in Table 3, it was seen that the mice produced effective HI antibody titers, and an average HI level could reach 2⁴ to 2⁸. Among them, PR8-M(sn)+Mut6+Del8 and PR8-M(sn)+Mut6+Del73 strains had excellent immune effects and could achieve complete protection. The attenuated strain PR8-M(sn)+Mut6+Del28 had obviously insufficient immunogenicity. In addition, the results in FIG. 6 showed that all strains could protect mice from virus infection after challenge. Except that the body weight of the mice in the PR8-M(sn)+Mut6+Del51 group changed greatly, the body weight of the mice in the PR8-M(sn)+Mut6+Del8 and PR8-M(sn)+Mut6+Del73 groups basically remained stable. It was seen that the PR8-M(sn)+Mut6+Del51 virus strain had the weakest toxicity. However, based on the results of the HI antibody titer and the challenge test, the immunogenicity of the PR8-M(sn)+Mut6+Del51 strain was weaker than that of the PR8-M(sn)+Mut6+Del8 and the PR8-M(sn)+Mut6+Del73. The above results further illustrated that the attenuated influenza virus of the present disclosure produced effective antibodies against influenza HA, but the attenuated viruses produced by different attenuation methods showed different immunogenicities.

In summary, the attenuated virus strains produced by the method of the present disclosure have desirable growth characteristics and can grow in MDCK-M2 and chicken embryos. Meanwhile, viruses with high-dose inoculation can proliferate in MDCK cells, showing the restricted replication ability of the virus. The attenuated virus has better immunogenicity than that of the single-replication virus. In addition, the different attenuated viruses produced by the method vary in virulence and immunogenicity. Among them, the strain with slower virus shedding is PR8-M(sn)+Mut6+Del8, and the strain with poor immunogenicity is PR8-M(sn)+Mut6+Del28. Based on the growth characteristics, toxicity, and immunogenicity data, it is known that the strain with better performance in the present disclosure is PR8-M(sn)+Mut6+Del73. According to different preparation uses of influenza vaccines, the method of the present disclosure can lay a foundation for the preparation of candidate strains of the attenuated influenza live vaccines and the attenuated and inactivated avian influenza vaccines.

The above are merely preferred embodiments of the present disclosure. It should be noted that several improvements and modifications may further be made by a person of ordinary skill in the art without departing from the principle of the present disclosure, and such improvements and modifications should also be deemed as falling within the protection scope of the present disclosure.

| Sequence Listing Information: | |
|---|---|
| DTD Version: V1_3 | |
| File Name: HLP20230402520_seqlist.xml | |
| Software Name: WIPO Sequence | |
| Software Version: 2.3.0 | |
| Production Date: 2023 May 26 | |
| General Information: | |
| Current application/Applicant file reference: HLP20230402520 | |
| Applicant name: Zhejiang Difference Biological Technology Co., Ltd. | |
| Applicant name/Language: en | |
| Invention title: METHOD FOR ATTENUATING INFLUENZA VIRUS BY | |
| SYNONYMOUS MUTATION AND DELETION MUTATION, AND ATTENUATED | |
| INFLUENZA VIRUS STRAIN AND USE (en) | |
| Sequence Total Quantity: 24 | |
| Sequences: | |
| Sequence Number (ID): 1 | |
| Length: 46 | |
| Molecule Type: DNA | |
| Features Location/Qualifiers: | |
| source, 1 .. 46 | |
| mol_type, other DNA | |
| organism, synthetic construct | |
| Residues: | |
| gcgtaccaaa agcgtatggg tgttcaaatg cagagattta aataag | 46 |
| Sequence Number (ID): 2 | |
| Length: 974 | |
| Molecule Type: DNA | |
| Features Location/Qualifiers: | |
| source, 1 .. 974 | |
| mol_type, other DNA | |
| organism, synthetic construct | |
| Residues: | |
| atgagtcttc taaccgaggt cgaaacgtac gtactctcta tcatcccgtc aggccccctc | 60 |
| aaagccgaga tcgcacagag acttgaagat gtctttgcag ggaagaacac cgatcttgag | 120 |
| gttctcatgg aatggctaaa gacaagacca atcctgtcac ctctgactaa ggggatttta | 180 |
| ggatttgtgt tcacgctcac cgtgcccagt gagcgaggac tgcagcgtag acgctttgtc | 240 |
| caaaatgccc ttaatgggaa cggggatcca aataacatgg acaaagcagt taaactgtat | 300 |
| aggaagctca agagggagat aacattccat ggggccaaag aaatctcact cagttattct | 360 |
| gctggtgcac ttgccagttg tatgggcctc atatacaaca ggatgggggc tgtgaccact | 420 |
| gaagtggcat ttggcctggt atgtgcaacc tgtgaacaga ttgctgactc ccagcatcgg | 480 |
| tctcataggc aaatggtgac aacaaccaat ccactaatca gacatgagaa cagaatggtt | 540 |
| ttagccagca ctacagctaa ggctatggag caaatggctg gatcgagtga gcaagcagca | 600 |
| gaggccatgg aggttgctag tcaggctaga caaatggtgc aagcgatgag aaccattggg | 660 |
| actcatccta gctccagtgc tggtctgaaa aatgatcttc ttgaaaattt gcaggcgtac | 720 |
| caaaagcgta tgggtgttca aatgcagaga tttaaataag tgatgacgca aatatcattg | 780 |
| ggatcttgca cttgacattg tggattcttg atcgtctttt tttcaaatgc atttaccgtc | 840 |
| gctttaaata cggactgaaa ggagggcctt ctacggaagg agtgccaaag tctatgaggg | 900 |
| aagaatatcg aaaggaacag cagagtgctg tggatgctga cgatggtcat tttgtcagca | 960 |
| tagagctgga gtaa | 974 |
| Sequence Number (ID): 3 | |
| Length: 954 | |
| Molecule Type: DNA | |
| Features Location/Qualifiers: | |
| source, 1 .. 954 | |
| mol_type, other DNA | |
| organism, synthetic construct | |
| Residues: | |
| atgagtcttc taaccgaggt cgaaacgtac gtactctcta tcatcccgtc aggccccctc | 60 |
| aaagccgaga tcgcacagag acttgaagat gtctttgcag ggaagaacac cgatcttgag | 120 |
| gttctcatgg aatggctaaa gacaagacca atcctgtcac ctctgactaa ggggatttta | 180 |
| ggatttgtgt tcacgctcac cgtgcccagt gagcgaggac tgcagcgtag acgctttgtc | 240 |
| caaaatgccc ttaatgggaa cggggatcca aataacatgg acaaagcagt taaactgtat | 300 |
| aggaagctca agagggagat aacattccat ggggccaaag aaatctcact cagttattct | 360 |
| gctggtgcac ttgccagttg tatgggcctc atatacaaca ggatgggggc tgtgaccact | 420 |
| gaagtggcat ttggcctggt atgtgcaacc tgtgaacaga ttgctgactc ccagcatcgg | 480 |
| tctcataggc aaatggtgac aacaaccaat ccactaatca gacatgagaa cagaatggtt | 540 |
| ttagccagca ctacagctaa ggctatggag caaatggctg gatcgagtga gcaagcagca | 600 |
| gaggccatgg aggttgctag tcaggctaga caaatggtgc aagcgatgag aaccattggg | 660 |
| actcatccta gctccagtgc tggtctgaaa aatgatcttc ttgaaaattt gcaggcgtac | 720 |
| caaaagcgta tgggtgttca aatgcagaga tttaaataag tgatgatgca cttgacattg | 780 |
| tggattcttg atcgtctttt tttcaaatgc atttaccgtc gctttaaata cggactgaaa | 840 |
| ggagggcctt ctacggaagg agtgccaaag tctatgaggg aagaatatcg aaaggaacag | 900 |
| cagagtgctg tggatgctga cgatggtcat tttgtcagca tagagctgga gtaa | 954 |
| Sequence Number (ID): 4 | |
| Length: 931 | |
| Molecule Type: DNA | |
| Features Location/Qualifiers: | |
| source, 1 .. 931 | |
| mol_type, other DNA | |
| organism, synthetic construct | |
| Residues: | |
| atgagtcttc taaccgaggt cgaaacgtac gtactctcta tcatcccgtc aggccccctc | 60 |
| aaagccgaga tcgcacagag acttgaagat gtctttgcag ggaagaacac cgatcttgag | 120 |
| gttctcatgg aatggctaaa gacaagacca atcctgtcac ctctgactaa ggggatttta | 180 |
| ggatttgtgt tcacgctcac cgtgcccagt gagcgaggac tgcagcgtag acgctttgtc | 240 |
| caaaatgccc ttaatgggaa cggggatcca aataacatgg acaaagcagt taaactgtat | 300 |
| aggaagctca agagggagat aacattccat ggggccaaag aaatctcact cagttattct | 360 |
| gctggtgcac ttgccagttg tatgggcctc atatacaaca ggatgggggc tgtgaccact | 420 |
| gaagtggcat ttggcctggt atgtgcaacc tgtgaacaga ttgctgactc ccagcatcgg | 480 |
| tctcataggc aaatggtgac aacaaccaat ccactaatca gacatgagaa cagaatggtt | 540 |
| ttagccagca ctacagctaa ggctatggag caaatggctg gatcgagtga gcaagcagca | 600 |
| gaggccatgg aggttgctag tcaggctaga caaatggtgc aagcgatgag aaccattggg | 660 |
| actcatccta gctccagtgc tggtctgaaa aatgatcttc ttgaaaattt gcaggcgtac | 720 |
| caaaagcgta tgggtgttca aatgcagaga tttaaataag tgatgagatc gtcttttttt | 780 |
| caaatgcatt taccgtcgct ttaaatacgg actgaaagga gggccttcta cggaaggagt | 840 |
| gccaaagtct atgagggaag aatatcgaaa ggaacagcag agtgctgtgg atgctgacga | 900 |
| tggtcatttt gtcagcatag agctggagta a | 931 |
| Sequence Number (ID): 5 | |
| Length: 909 | |
| Molecule Type: DNA | |
| Features Location/Qualifiers: | |
| source, 1 .. 909 | |
| mol_type, other DNA | |
| organism, synthetic construct | |
| Residues: | |
| atgagtcttc taaccgaggt cgaaacgtac gtactctcta tcatcccgtc aggccccctc | 60 |
| aaagccgaga tcgcacagag acttgaagat gtctttgcag ggaagaacac cgatcttgag | 120 |
| gttctcatgg aatggctaaa gacaagacca atcctgtcac ctctgactaa ggggatttta | 180 |
| ggatttgtgt tcacgctcac cgtgcccagt gagcgaggac tgcagcgtag acgctttgtc | 240 |
| caaaatgccc ttaatgggaa cggggatcca aataacatgg acaaagcagt taaactgtat | 300 |
| aggaagctca agagggagat aacattccat ggggccaaag aaatctcact cagttattct | 360 |
| gctggtgcac ttgccagttg tatgggcctc atatacaaca ggatgggggc tgtgaccact | 420 |
| gaagtggcat ttggcctggt atgtgcaacc tgtgaacaga ttgctgactc ccagcatcgg | 480 |
| tctcataggc aaatggtgac aacaaccaat ccactaatca gacatgagaa cagaatggtt | 540 |
| ttagccagca ctacagctaa ggctatggag caaatggctg gatcgagtga gcaagcagca | 600 |
| gaggccatgg aggttgctag tcaggctaga caaatggtgc aagcgatgag aaccattggg | 660 |
| actcatccta gctccagtgc tggtctgaaa aatgatcttc ttgaaaattt gcaggcgtac | 720 |
| caaaagcgta tgggtgttca aatgcagaga tttaaataag tgatgattta ccgtcgcttt | 780 |
| aaatacggac tgaaaggagg gccttctacg gaaggagtgc caaagtctat gagggaagaa | 840 |
| tatcgaaagg aacagcagag tgctgtggat gctgacgatg gtcattttgt cagcatagag | 900 |
| ctggagtaa | 909 |
| Sequence Number (ID): 6 | |
| Length: 871 | |
| Molecule Type: DNA | |
| Features Location/Qualifiers: | |
| source, 1 .. 871 | |
| mol_type, other DNA | |
| organism, synthetic construct | |
| Residues: | |
| atgagtcttc taaccgaggt cgaaacgtac gtactctcta tcatcccgtc aggccccctc | 60 |
| aaagccgaga tcgcacagag acttgaagat gtctttgcag ggaagaacac cgatcttgag | 120 |
| gttctcatgg aatggctaaa gacaagacca atcctgtcac ctctgactaa ggggatttta | 180 |
| ggatttgtgt tcacgctcac cgtgcccagt gagcgaggac tgcagcgtag acgctttgtc | 240 |
| caaaatgccc ttaatgggaa cggggatcca aataacatgg acaaagcagt taaactgtat | 300 |
| aggaagctca agagggagat aacattccat ggggccaaag aaatctcact cagttattct | 360 |
| gctggtgcac ttgccagttg tatgggcctc atatacaaca ggatgggggc tgtgaccact | 420 |
| gaagtggcat ttggcctggt atgtgcaacc tgtgaacaga ttgctgactc ccagcatcgg | 480 |
| tctcataggc aaatggtgac aacaaccaat ccactaatca gacatgagaa cagaatggtt | 540 |
| ttagccagca ctacagctaa ggctatggag caaatggctg gatcgagtga gcaagcagca | 600 |
| gaggccatgg aggttgctag tcaggctaga caaatggtgc aagcgatgag aaccattggg | 660 |
| actcatccta gctccagtgc tggtctgaaa aatgatcttc ttgaaaattt gcaggcgtac | 720 |
| caaaagcgta tgggtgttca aatgcagaga tttaaataag tgatgatcta cggaaggagt | 780 |
| gccaaagtct atgagggaag aatatcgaaa ggaacagcag agtgctgtgg atgctgacga | 840 |
| tggtcatttt gtcagcatag agctggagta a | 871 |
| Sequence Number (ID): 7 | |
| Length: 38 | |
| Molecule Type: DNA | |
| Features Location/Qualifiers: | |
| source, 1 .. 38 | |
| mol_type, other DNA | |
| organism, synthetic construct | |
| Residues: | |
| tcaagtgatt gatgacgcaa atatcattgg gatcttgc | 38 |
| Sequence Number (ID): 8 | |
| Length: 22 | |
| Molecule Type: DNA | |
| Features Location/Qualifiers: | |
| source, 1 .. 22 | |
| mol_type, other DNA | |
| organism, synthetic construct | |
| Residues: | |
| tcatcaatca cttgaaccgt tg | 22 |
| Sequence Number (ID): 9 | |
| Length: 39 | |
| Molecule Type: DNA | |
| Features Location/Qualifiers: | |
| source, 1 .. 39 | |
| mol_type, other DNA | |
| organism, synthetic construct | |
| Residues: | |
| tcaagtgatt gatgatgcac ttgacattgt ggattcttg | 39 |
| Sequence Number (ID): 10 | |
| Length: 22 | |
| Molecule Type: DNA | |
| Features Location/Qualifiers: | |
| source, 1 .. 22 | |
| mol_type, other DNA | |
| organism, synthetic construct | |
| Residues: | |
| tcatcaatca cttgaaccgt tg | 22 |
| Sequence Number (ID): 11 | |
| Length: 35 | |
| Molecule Type: DNA | |
| Features Location/Qualifiers: | |
| source, 1 .. 35 | |
| mol_type, other DNA | |
| organism, synthetic construct | |
| Residues: | |
| tcaagtgatt gatgagatcg tctttttttc aaatg | 35 |
| Sequence Number (ID): 12 | |
| Length: 22 | |
| Molecule Type: DNA | |
| Features Location/Qualifiers: | |
| source, 1 .. 22 | |
| mol_type, other DNA | |
| organism, synthetic construct | |
| Residues: | |
| tcatcaatca cttgaaccgt tg | 22 |
| Sequence Number (ID): 13 | |
| Length: 36 | |
| Molecule Type: DNA | |
| Features Location/Qualifiers: | |
| source, 1 .. 36 | |
| mol_type, other DNA | |
| organism, synthetic construct | |
| Residues: | |
| tcaagtgatt gatgatttac cgtcgcttta aatacg | 36 |
| Sequence Number (ID): 14 | |
| Length: 22 | |
| Molecule Type: DNA | |
| Features Location/Qualifiers: | |
| source, 1 .. 22 | |
| mol_type, other DNA | |
| organism, synthetic construct | |
| Residues: | |
| tcatcaatca cttgaaccgt tg | 22 |
| Sequence Number (ID): 15 | |
| Length: 36 | |
| Molecule Type: DNA | |
| Features Location/Qualifiers: | |
| source, 1 .. 36 | |
| mol_type, other DNA | |
| organism, synthetic construct | |
| Residues: | |
| tcaagtgatt gatgatctac ggaaggagtg ccaaag | 36 |
| Sequence Number (ID): 16 | |
| Length: 22 | |
| Molecule Type: DNA | |
| Features Location/Qualifiers: | |
| source, 1 .. 22 | |
| mol_type, other DNA | |
| organism, synthetic construct | |
| Residues: | |
| tcatcaatca cttgaaccgt tg | 22 |
| Sequence Number (ID): 17 | |
| Length: 46 | |
| Molecule Type: DNA | |
| Features Location/Qualifiers: | |
| source, 1 .. 46 | |
| mol_type, other DNA | |
| organism, synthetic construct | |
| Residues: | |
| gcctatcaga aacgaatggg ggtgcagatg caacggttca agtgat | 46 |
| Sequence Number (ID): 18 | |
| Length: 28 | |
| Molecule Type: DNA | |
| Features Location/Qualifiers: | |
| source, 1 .. 28 | |
| mol_type, other DNA | |
| organism, synthetic construct | |
| Residues: | |
| actattgccg caaatatcat tgggatct | 28 |
| Sequence Number (ID): 19 | |
| Length: 51 | |
| Molecule Type: DNA | |
| Features Location/Qualifiers: | |
| source, 1 .. 51 | |
| mol_type, other DNA | |
| organism, synthetic construct | |
| Residues: | |
| actattgccg caaatatcat tgggatcttg cacttgacat tgtggattct t | 51 |
| Sequence Number (ID): 20 | |
| Length: 73 | |
| Molecule Type: DNA | |
| Features Location/Qualifiers: | |
| source, 1 .. 73 | |
| mol_type, other DNA | |
| organism, synthetic construct | |
| Residues: | |
| actattgccg caaatatcat tgggatcttg cacttgacat tgtggattct tgatcgtctt | 60 |
| tttttcaaat gca | 73 |
| Sequence Number (ID): 21 | |
| Length: 111 | |
| Molecule Type: DNA | |
| Features Location/Qualifiers: | |
| source, 1 .. 111 | |
| mol_type, other DNA | |
| organism, synthetic construct | |
| Residues: | |
| actattgccg caaatatcat tgggatcttg cacttgacat tgtggattct tgatcgtctt | 60 |
| tttttcaaat gcatttaccg tcgctttaaa tacggactga aaggagggcc t | 111 |
| Sequence Number (ID): 22 | |
| Length: 52 | |
| Molecule Type: DNA | |
| Features Location/Qualifiers: | |
| source, 1 .. 52 | |
| mol_type, other DNA | |
| organism, synthetic construct | |
| Residues: | |
| atgggtgttc aaatgcagag atttaaataa gcctctcact attgccgcaa at | 52 |
| Sequence Number (ID): 23 | |
| Length: 22 | |
| Molecule Type: DNA | |
| Features Location/Qualifiers: | |
| source, 1 .. 22 | |
| mol_type, other DNA | |
| organism, synthetic construct | |
| Residues: | |
| gagaggctta tttaaatctc tg | 22 |
| Sequence Number (ID): 24 | |
| Length: 982 | |
| Molecule Type: DNA | |
| Features Location/Qualifiers: | |
| source, 1 .. 982 | |
| mol_type, other DNA | |
| organism, synthetic construct | |
| Residues: | |
| atgagtcttc taaccgaggt cgaaacgtac gtactctcta tcatcccgtc aggccccctc | 60 |
| aaagccgaga tcgcacagag acttgaagat gtctttgcag ggaagaacac cgatcttgag | 120 |
| gttctcatgg aatggctaaa gacaagacca atcctgtcac ctctgactaa ggggatttta | 180 |
| ggatttgtgt tcacgctcac cgtgcccagt gagcgaggac tgcagcgtag acgctttgtc | 240 |
| caaaatgccc ttaatgggaa cggggatcca aataacatgg acaaagcagt taaactgtat | 300 |
| aggaagctca agagggagat aacattccat ggggccaaag aaatctcact cagttattct | 360 |
| gctggtgcac ttgccagttg tatgggcctc atatacaaca ggatgggggc tgtgaccact | 420 |
| gaagtggcat ttggcctggt atgtgcaacc tgtgaacaga ttgctgactc ccagcatcgg | 480 |
| tctcataggc aaatggtgac aacaaccaat ccactaatca gacatgagaa cagaatggtt | 540 |
| ttagccagca ctacagctaa ggctatggag caaatggctg gatcgagtga gcaagcagca | 600 |
| gaggccatgg aggttgctag tcaggctaga caaatggtgc aagcgatgag aaccattggg | 660 |
| actcatccta gctccagtgc tggtctgaaa aatgatcttc ttgaaaattt gcaggcctat | 720 |
| cagaaacgaa tgggggtgca gatgcaacgg ttcaagtgat cctctcacta ttgccgcaaa | 780 |
| tatcattggg atcttgcact tgacattgtg gattcttgat cgtctttttt tcaaatgcat | 840 |
| ttaccgtcgc tttaaatacg gactgaaagg agggccttct acggaaggag tgccaaagtc | 900 |
| tatgagggaa gaatatcgaa aggaacagca gagtgctgtg gatgctgacg atggtcattt | 960 |
| tgtcagcata gagctggagt aa | 982 |
| END | |

What is claimed is:

1. A method for attenuating an influenza virus, comprising:
a) conducting synonymous mutation on an overlap of an M2 gene and an M1 gene in an M gene of an influenza A virus, while ensuring integrity and invariance of an amino acid sequence encoded by the M1 gene:
b) conducting mutation into termination codon and deletion of partial nucleotide sequence in a transmembrane domain of the M2 gene, and
c) rescuing an attenuated influenza virus strain using a reverse genetic system.

2. The method according to claim 1, wherein a background strain from which the M gene derives comprises A/Puerto Rico/8/1934.

3. The method according to claim 1, wherein the synonymous mutation comprises: conducting mutation on the 715th base to the 760th base of an M gene of SEQ ID NO: 24 into a nucleotide sequence of SEQ ID NO: 1.

4. The method according to claim 1, wherein the mutation into termination codon comprises:
conducting mutation on the 761th base to the 766th base of an M gene of SEQ ID NO: 24 into the nucleotide sequence of TGATGA.

5. The method according to claim 1, wherein the deletion of partial nucleotide sequence comprises: conducting deletion on a nucleotide sequence of any position and length from the 767th base to the 877th base of an M gene of SEQ ID NO: 24.

6. The method according to claim 5, wherein the deletion of partial nucleotide sequence specifically comprises:
conducting deletion on the 767th base to the 774th base of the M gene of SEQ ID NO: 24:
conducting deletion on the 767th base to the 794th base of the M gene of SEQ ID NO: 24:
conducting deletion on the 767th base to the 817th base of the M gene of SEQ ID NO: 24:
conducting deletion on the 767th base to the 839th base of the M gene of SEQ ID NO: 24: or conducting deletion on the 767th base to the 877th base of the M gene of SEQ ID NO: 24.

7. An attenuated influenza virus strain prepared by the method according to claim 1.

8. The attenuated influenza virus strain according to claim 7, wherein a background strain from which the M gene derives comprises A/Puerto Rico/8/1934.

9. The attenuated influenza virus strain according to claim 7, wherein the synonymous mutation comprises: conducting mutation on the 715th base to the 760th base of an M gene of SEQ ID NO: 24 into a nucleotide sequence of SEQ ID NO: 1.

10. The attenuated influenza virus strain according to claim 7, wherein the mutation into termination codon comprises: conducting mutation on the 761th base to the 766th base of an M gene of SEQ ID NO: 4 into a nucleotide sequence of TGATGA.

11. The attenuated influenza virus strain according to claim 7, wherein the deletion of partial nucleotide sequence comprises: conducting deletion on a nucleotide sequence of any position and length from the 767th base to the 877th base of an M gene of SEQ ID NO: 24.

12. The attenuated influenza virus strain according to claim 11, wherein the deletion of partial nucleotide sequence specifically comprises:
conducting deletion on the 767th base to the 774th base of the M gene of SEQ ID NO: 24:
conducting deletion on the 767th base to the 794th base of the M gene of SEQ ID NO: 24:
conducting deletion on the 767th base to the 817th base of the M gene of SEQ ID NO: 24:
conducting deletion on the 767th base to the 839th base of the M gene of SEQ ID NO: 24: or conducting deletion on the 767th base to the 877th base of the M gene of SEQ ID NO: 24.

13. The attenuated influenza virus strain according to claim 7, wherein the attenuated influenza virus strain takes the A/Puerto Rico/8/1934 as a background strain from which the M gene derives.

14. A set of defective plasmids for preparing the attenuated influenza virus strain according to claim 7, wherein the defective plasmids comprise the M gene of the influenza virus with the synonymous mutation, the mutation into termination codon, and the deletion of partial nucleotide sequence.

15. The set of defective plasmids according to claim 14, wherein the attenuated influenza virus strain takes the A/Puerto Rico/8/1934 as a background strain from which the M gene derives.


SEQUENCE LISTING (24 sequences)

SEQ ID NO: 1 - DNA, 46 bp, synthetic construct
  source 1..46: mol_type = other DNA
gcgtaccaaaagcgtatgggtgttcaaatgcagagatttaaataag

SEQ ID NO: 2 - DNA, 974 bp, synthetic construct
  source 1..974: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcgtaccaaaagcgtatgggtgttcaaatgcagagatttaaataagtgatgacgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 3 - DNA, 954 bp, synthetic construct
  source 1..954: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcgtaccaaaagcgtatgggtgttcaaatgcagagatttaaataagtgatgatgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 4 - DNA, 931 bp, synthetic construct
  source 1..931: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcgtaccaaaagcgtatgggtgttcaaatgcagagatttaaataagtgatgagatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 5 - DNA, 909 bp, synthetic construct
  source 1..909: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcgtaccaaaagcgtatgggtgttcaaatgcagagatttaaataagtgatgatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 6 - DNA, 871 bp, synthetic construct
  source 1..871: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcgtaccaaaagcgtatgggtgttcaaatgcagagatttaaataagtgatgatctacggaaggagtgccaaagtctatgagggaagaatatcgaaaggaacagcagagtgctgtggatgctgacgatggtcattttgtcagcatagagctggagtaa

SEQ ID NO: 7 - DNA, 38 bp, synthetic construct
  source 1..38: mol_type = other DNA
tcaagtgattgatgacgcaaatatcattgggatcttgc

SEQ ID NO: 8 - DNA, 22 bp, synthetic construct
  source 1..22: mol_type = other DNA
tcatcaatcacttgaaccgttg

SEQ ID NO: 9 - DNA, 39 bp, synthetic construct
  source 1..39: mol_type = other DNA
tcaagtgattgatgatgcacttgacattgtggattcttg

SEQ ID NO: 10 - DNA, 22 bp, synthetic construct
  source 1..22: mol_type = other DNA
tcatcaatcacttgaaccgttg

SEQ ID NO: 11 - DNA, 35 bp, synthetic construct
  source 1..35: mol_type = other DNA
tcaagtgattgatgagatcgtctttttttcaaatg

SEQ ID NO: 12 - DNA, 22 bp, synthetic construct
  source 1..22: mol_type = other DNA
tcatcaatcacttgaaccgttg

SEQ ID NO: 13 - DNA, 36 bp, synthetic construct
  source 1..36: mol_type = other DNA
tcaagtgattgatgatttaccgtcgctttaaatacg

SEQ ID NO: 14 - DNA, 22 bp, synthetic construct
  source 1..22: mol_type = other DNA
tcatcaatcacttgaaccgttg

SEQ ID NO: 15 - DNA, 36 bp, synthetic construct
  source 1..36: mol_type = other DNA
tcaagtgattgatgatctacggaaggagtgccaaag

SEQ ID NO: 16 - DNA, 22 bp, synthetic construct
  source 1..22: mol_type = other DNA
tcatcaatcacttgaaccgttg

SEQ ID NO: 17 - DNA, 46 bp, synthetic construct
  source 1..46: mol_type = other DNA
gcctatcagaaacgaatgggggtgcagatgcaacggttcaagtgat

SEQ ID NO: 18 - DNA, 28 bp, synthetic construct
  source 1..28: mol_type = other DNA
actattgccgcaaatatcattgggatct

SEQ ID NO: 19 - DNA, 51 bp, synthetic construct
  source 1..51: mol_type = other DNA
actattgccgcaaatatcattgggatcttgcacttgacattgtggattctt

SEQ ID NO: 20 - DNA, 73 bp, synthetic construct
  source 1..73: mol_type = other DNA
actattgccgcaaatatcattgggatcttgcacttgacattgtggattcttgatcgtctttttttcaaatgca

SEQ ID NO: 21 - DNA, 111 bp, synthetic construct
  source 1..111: mol_type = other DNA
actattgccgcaaatatcattgggatcttgcacttgacattgtggattcttgatcgtctttttttcaaatgcatttaccgtcgctttaaatacggactgaaaggagggcct

SEQ ID NO: 22 - DNA, 52 bp, synthetic construct
  source 1..52: mol_type = other DNA
atgggtgttcaaatgcagagatttaaataagcctctcactattgccgcaaat

SEQ ID NO: 23 - DNA, 22 bp, synthetic construct
  source 1..22: mol_type = other DNA
gagaggcttatttaaatctctg

SEQ ID NO: 24 - DNA, 982 bp, synthetic construct
  source 1..982: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa